(19) United States
(12) Patent Application Publication
(10) Pub. No.: US 2018/0135111 A1
(43) Pub. Date: May 17, 2018

(54) METHOD AND APPARATUS FOR ULTRASENSITIVE QUANTITATIVE ANALYSIS OF DNA BIOMARKER

(71) Applicant: POSTECH ACADEMY-INDUSTRY FOUNDATION, Pohang-si (KR)

(72) Inventors: Joon Won Park, Seoul (KR); Yoonhee Lee, Seoul (KR); Youngkyu Kim, New York, NY (US); Donggyu Lee, Busan (KR)

(21) Appl. No.: 15/635,393

(22) Filed: Jun. 28, 2017

(30) Foreign Application Priority Data
Nov. 16, 2016 (KR) 10-2016-0152895

(51) Int. Cl. C12Q 1/68; G01Q 60/42

(52) U.S. Cl. CPC C12Q 1/6816; G01Q 60/42; C12Q 1/6834

(57) ABSTRACT

The present invention relates to a method and an apparatus for ultrasensitive quantitative analysis of a DNA biomarker, and more particularly, provides a method and a kit for quantitative analysis of target DNA using an atomic force microscope. The DNA quantification according to the present invention enables the quantification of target DNA at low concentration, which has been difficult to carry out by a conventional method, through adhesion force mapping by using an atomic force microscope, and does not result in DNA amplification and transformation nor require the use of a fluorescent marker. In particular, the method applies the size optimization of a probe DNA spot generated to capture target DNA, thereby enabling the quantitative analysis of ten or less target DNA molecules on various substrates. When applied for diagnosing a disease, monitoring the prognosis of treatment, and the like, the method for ultrasensitive quantitative analysis of DNA according to the present invention is expected to be applicable not only to the early diagnosis of a disease but also to monitoring the progress of surgery and treatment.

### CROSS-REFERENCE TO RELATED APPLICATION

This application claims priority to and the benefit of Korean Patent Application No. 10-2016-0152895, filed on Nov. 16, 2016, the disclosure of which is incorporated herein by reference in its entirety.

### BACKGROUND

### 1. Field of the Invention

The present invention relates to a method and an apparatus for ultrasensitive quantitative analysis of a DNA biomarker. More particularly, the present invention provides a method and a kit for quantitative analysis of target DNA using an atomic force microscope.

### 2. Discussion of Related Art

In modern medicine, identifying the genetic nature and characteristics of a disease of unknown origin is an essential element for accurate diagnosis. In particular, molecular diagnostic technology identifies a mutation, which is an origin of a disease, in patient's DNA and RNA samples by gene sequence information, and thus can provide important information for accurately diagnosing a cause of the disease before the disease progresses and estimating a prognosis thereof. Also, molecular diagnostics is used for studying the mechanism of gene expression and functions and thus is positioned as an important field that provides basic knowledge in drug research and biomarker development.

Currently, the most powerful molecular diagnostic tool is polymerase chain reaction (PCR), which was first reported in 1985 (Saiki et al. Science 230, 1350-1354 (1985)). PCR is capable of identifying a target mutation within a few days by amplifying a specific region of DNA in a selective manner using a specific enzyme. In particular, as an additional aid to a conventional PCR method, quantitative real-time polymerase chain reaction (qPCR), which was devised for DNA amplification and simultaneous DNA detection. It employs a fluorescent marker which detects an amplification event of the specific target DNA and tracks amplification cycles. qPCR, which is a method of inferring an unknown sample amount based on an amplification curve of a standard material, is theoretically capable of quantification to a single-molecule level but has difficulty in the analysis due to a large deviation in analysis results. Also, an amplification error such as the formation of primer dimers may lead to a false positive result (Hughes et al. Lancet 335, 1037-1038 (1990)), DNA amplification efficiency may vary depending on a nucleic acid structure, and quantitative deviation may occur due to an error caused while correcting a standard material and a target DNA (Sanders et al. Anal. Bioanal. Chem. 406, 6471-6483 (2014)).

One of the alternatives to overcome the problem of qPCR is digital PCR (dPCR) (Sykes et al. Biotechniques 13, 444-449 (1992)). dPCR distributes target DNA molecules in nanoliter-volume compartments in a statistical manner such that each compartment contains zero or one target DNA molecule, performs parallel amplification of the DNA molecules, and then calculates the number of compartments having a positive or negative PCR amplification result so as to determine the initial concentration of the target DNA. Since there is no need to use a standard material to obtain an amplification curve, an error caused due to a difference of amplification efficiency can be eliminated (Hindson et al. Nature Methods 10, 1003-1005 (2013)). However, a measurement error occurs due to nonuniform compartment volume or compartments containing two or more target DNA molecules. Also, the infinitesimally smaller the amount of the target DNA is, the greater the impact that the measurement error has on the result (Corbisier et al. Anal. Bioanal. Chem. 407, 1831-1840 (2015)).

Owing to such problems, research on methods of accurately calculating target DNA at low concentration without the use of PCR has been conducted in recent years, and single-molecule force spectroscopy using an atomic force microscope is one of the methods. An atomic force microscope has nanometer-scale spatial resolution and high sensitivity that enables the measurement of interaction forces between a cantilever and a surface up to several piconewtons (pN). A tip at the end of the cantilever is several nanometers in radius, and the desired biomolecule may be introduced onto the tip by way of chemically treating the tip surface. In this way, interaction forces between two single biomolecules (DNA-DNA, DNA-RNA, antigen-antibody, protein-ligand, etc.) can be measured, and the molecular structure, surface distribution, dynamics, can be analyzed based on the measurement (Hinterdorfer et al. Nat. Methods 3, 347-355 (2006)). In particular, much research based on a force mapping method has been conducted to measure the concentration of an antigen or target DNA captured on a surface onto which an antibody or probe DNA was introduced, wherein the force mapping method programs a cantilever move pixel by pixel to collect the force-distance curves at the nanometer spaial resolution, and thereby obtains a quantitative image of adhesion force, Young's modulus, and height. U.S. Pat. No. 8,067,169 proposed a method of detecting a short nucleic acid on a flat, solid surface using an atomic force microscope and a T-shaped cantilever. The method measures initial microRNA concentration based on a difference in stiffness between single-strand probe DNA and a DNA-RNA dimer that is formed when a surface containing immobilized single-strand probe DNA is treated with target microRNA. Also, U.S. Pat. No. 7,152,462 proposed topography and recognition imaging (TREC) that enables the simultaneous observation of a surface topographical image and a molecular recognition image in a short time. TREC uses a cantilever that includes biotin molecules on a tip thereof to simultaneously obtain a topographic image of a surface including avidin distributed thereon and a molecular recognition image of biotin-avidin. By comparing the above images, TREC can improve the position accuracy of an individual avidin molecule and measure molecular activity.

However, there are two major problems in devising an apparatus for quantitative analysis using the above methods. First, molecules in an image are obtained without defining the feature size of a single molecule. Therefore, when two or more target molecules are distributed close to one another on a surface, individual molecular images may be overlapped, making it difficult to count the number of such images, thus leading to a quantitative error. Also, target molecules should be collected intensively on a surface. The spatial resolution should be maintained within a range of several nanometers to several tens of nanometers to obtain a target molecule image. Therefore, probe molecules are printed on the surface, commonly by using a microarray device, to attract target molecules to specific regions and capture the same molecules within the specific regions. In this case, the probe spot diameter ranges from several tens of micrometers (microns) to several hundreds of microns. Since the maximum resolution of a quantitative image obtained by a general atomic force microscope is limited to 512×512 pixels, not only it is impossible to image an entire microarray spot in nanometer scale but also the durability of the cantilever may be negatively affected as more force-distance curves are being collected. For this reason, the number of target molecules dispersed on the entire spot has been estimated by scanning a part of a microarray spot.

As a result of efforts put into developing a method of quantitatively analyzing DNA molecules with high accuracy, the present inventors have confirmed that single DNA presented on the surface can be counted when a spot where the probe molecules are immobilized can be printed into a size of ten microns or less. Because the spot size is capable of being scanned at the nanometer-scale spatial resolution required for obtaining a molecular image and thereby the present inventors have completed the present invention.

### SUMMARY OF THE INVENTION

The present invention is directed to providing a kit for quantitatively analyzing target DNA, the kit including: a cantilever for an atomic force microscope, wherein the cantilever includes a tip at the end of a body thereof and detection DNA immobilized to a surface of the tip, and the detection DNA contains base sequences that can be complementarily bound to target DNA; and a printing substrate to which probe DNA is immobilized, wherein the probe DNA contains base sequences that can be complementarily bound to the target DNA.

Also, the present invention is directed to providing a method of manufacturing a printing substrate for quantitatively analyzing target DNA, the method including: a process of preparing a printing substrate, a process of etching a pattern with an area of 10 to 900 μm² and a depth of 10 to 900 nm on the printing substrate, and a process of printing a probe DNA spot by applying, on the etched pattern, a drop of an aqueous probe DNA solution containing base sequences capable of complementarily binding to the target DNA.

In addition, the present invention is directed to providing a method of quantitatively analyzing target DNA, the method including: (a) a hybridization process that forms DNA/DNA dimers by applying a sample containing target DNA on a printing substrate that includes probe DNA printed thereon, wherein the probe DNA contains base sequences capable of complementarily binding to the target DNA; (b) a process of bringing a cantilever for an atomic force microscope, which includes detection DNA containing base sequences capable of complementarily binding to the target DNA, into contact with the printing substrate including the DNA/DNA dimers formed thereon to conduct adhesion force mapping within a probe DNA spot; and (c) a process of detecting the presence of the DNA/DNA dimers, which were formed between the probe DNA and target DNA, in a spot where an adhesion force is observed on the adhesion force map, and analyzing the spatial distribution of the captured DNAs.

The present invention is devised to solve the aforementioned problems, and provides a cantilever for an atomic force microscope, wherein the cantilever includes a tip at the end of a body thereof and detection DNA immobilized to a surface of the tip, and the detection DNA contains base sequences capable of complementarily binding to target DNA; and a kit for quantitatively analyzing target DNA, wherein the kit includes a printing substrate to which probe DNA is immobilized, and the probe DNA contains base sequences that can be complementarily bound to the target DNA. In the present invention, the probe DNA may be printed as a spot with a diameter of 100 nm to 10 μm on the printing substrate.

The probe DNA according to the present invention may contain an amine group at a 3′-terminal thereof.

The printing substrate according to the present invention may be selected from the group consisting of glass, metals, plastics, silicon, silicates, ceramic, semiconductors, synthetic organic metals, synthetic semiconductors, and alloys.

The detection DNA according to the present invention may contain an amine group at a 5′-terminal thereof.

The kit according to the present invention may further include an atomic force microscope.

Also, the present invention provides a method of manufacturing a printing substrate for quantitatively analyzing target DNA, the method including: a process of preparing a printing substrate, a process of etching a pattern with an area of 10 to 900 μm² and a depth of 10 to 900 nm on the printing substrate, and a process of printing a probe DNA spot by applying, on the etched pattern, a drop of an aqueous probe DNA solution containing base sequences capable of complementarily binding to the target DNA.

The printing of the probe DNA according to the present invention may be a process of dropping an aqueous probe DNA solution forming a probe DNA spot with a diameter of 100 nm to 10 μm on the printing substrate.

The probe DNA according to the present invention may contain an amine group at a 3′-terminal thereof.

The printing substrate according to the present invention may be selected from the group consisting of glass, metals, plastics, silicon, silicates, ceramic, semiconductors, synthetic organic metals, synthetic semiconductors, and alloys.

The method of manufacturing a printing substrate for quantitatively analyzing target DNA according to the present invention may further include a process of amine functionalization onto the substrate surface after etching the substrate, and a process of introducing a bifunctional linker that forms a covalent bond with the amine functional group.

In addition, the present invention provides a method of quantitatively analyzing target DNA, the method including: (a) a hybridization process that forms DNA/DNA dimers by applying a sample containing target DNA on a printing substrate that includes probe DNA printed thereon, wherein the probe DNA contains base sequences capable of complementarily binding to the target DNA; (b) a process of bringing a cantilever for an atomic force microscope, which includes detection DNA containing base sequences capable of complementarily binding to the target DNA, into contact with the printing substrate including the DNA/DNA dimers formed thereon to conduct adhesion force mapping within a probe DNA spot; and (c) a process of detecting the presence of the DNA/DNA dimers, which were formed between the probe DNA and target DNA, in a spot where an adhesion force is observed on the adhesion force map, and analyzing the spatial distribution of the captured DNAs.

In the present invention, the sample containing target DNA may contain the target DNA at a concentration of 10 zM to 1 aM.

The printing substrate that includes probe DNA printed thereon according to the present invention may include the target DNA immobilized in the form of a spot with a diameter of 100 nm to 10 μm onto a surface thereof.

The adhesion force mapping as a part of the process (b) according to the present invention may be conducted by measuring a specific force-distance curve obtained as a result of an interaction between the detection DNA and the target DNA, which takes place where the cantilever for an atomic force microscope contacts.

The analysis of spatial distribution of the captured DNAs as a part of the process (c) according to the present invention may be conducted by counting the number of DNA/DNA dimers with an observed adhesion force in the sample containing the target DNA.

Also, a method of counting the number of the captured DNAs with an observed adhesion force may include: a process of obtaining multiple adhesion force maps from the same probe DNA spot in a consecutive manner; a process of isolating clusters in which a specific adhesion force is observed in two or more adjacent pixels when multiple adhesion force maps are overlaid; a process of isolating, among the above clusters, clusters in which the specific adhesion force is repeatedly observed twice or more in the same pixels; and a process of counting, among pixel clusters resulting from overlaying of multiple pixels, clusters with a diameter greater than the diameter of a single target DNA cluster.

The method of quantitatively analyzing target DNA according to the present invention may further include determining a concentration of the target DNA after the completion of the process (c).

The method of quantitatively analyzing DNA according to the present invention enables the quantification of target DNA at low concentration, which has been difficult to carry out by conventional methods, through adhesion force mapping by using an atomic force microscope. This method does not result in DNA amplification and transformation nor requires the use of a fluorescent marker. In particular, the method optimizes the size of probe DNA spot capturing target DNA, thereby enables the quantitative analysis of ten or less target DNA molecules on various substrates.

When applied for diagnosing a disease, monitoring the prognosis of treatment, and the like, the method for ultrasensitive quantitative analysis of DNA according to the present invention is expected to be applicable not only to the early diagnosis of a disease but also to monitoring the progress of surgery and treatment.

### BRIEF DESCRIPTION OF THE DRAWINGS

The above and other objects, features, and advantages of the present invention will become more apparent to those of ordinary skill in the art by describing in detail exemplary embodiments thereof concerning the accompanying drawings, in which:

FIG. 1 is a schematic diagram for illustrating the principle of analysis of target DNA through an atomic force microscope according to the present invention;

FIG. 2 is a schematic diagram for illustrating the detection range of target DNA calculated based on a probe DNA spot diameter according to one exemplary embodiment of the present invention;

FIG. 3 is a set of images including (a) an image of etched patterns on a glass slide and (b) an image of a probe DNA spot printed on a specific square pattern among the etched patterns;

FIG. 4 is a set of images including (a) a schematic diagram for illustrating how detection DNA immobilized on a cantilever tip recognizes target DNA when the target DNA bound to a surface of a probe DNA spot exhibits hydrodynamic motion in an aqueous solution, (b) histograms of adhesion force values and stretching distance values measured from force-distance curves obtained in a cluster, and (c) images showing the shapes and measured radii of clusters observed in an adhesion force map;

FIG. 5 illustrates a process of overlaying three consecutive images obtained from the same probe DNA spot after compensating two-dimensional position differences between the images. Force-distance curves are collected from the entire spot surface, followed by (a) generating three consecutive images containing information on height and Young's modulus, (b) measuring the position difference between the first and second images and between the second and third images, and then (c) finally adjusting positions of the three images to obtain a quantitative image resulting from overlaying the three images;

FIG. 6 shows the location and frequency at which a specific adhesion force is measured in an adhesion force map obtained by the overlaying of images through position adjustment. (a) is a set of images including three maps showing the detected specific adhesion force obtained in a probe DNA spot treated with a 1 aM aqueous target DNA solution, an adhesion force frequency map obtained by overlaying the above maps after a position adjustment, wherein locations of valid clusters are marked with yellow circles. (b) is a set of images including overlaid adhesion force frequency maps obtained from a probe DNA spot not treated with any of target DNA molecules, wherein the location of the biggest cluster observed under the same conditions is marked with a white square; and

FIG. 7 shows quantitative analysis results of ten or less target DNA molecules. (a) is a graph showing a linear relationship between the number of target DNA molecules in a sample and the mean number of valid clusters observed in adhesion force maps, and (b) shows a histogram of radii of valid clusters observed in all samples and the mean value of the radii.

### DETAILED DESCRIPTION OF EXEMPLARY EMBODIMENTS

From now on, various exemplary embodiments of the present invention will be described. The present invention may be variously modified and have a variety of exemplary embodiments, and the following specific embodiments are merely illustrative and not intended to limit the present invention to the specific embodiments. It should be understood that the present invention covers various modifications, equivalents, and substitutions included in the scope and spirit of the present invention.

The present invention provides a kit for quantitatively analyzing target DNA, the kit including: a cantilever for an atomic force microscope, wherein the cantilever includes a tip at the end of a body thereof and detection DNA immobilized to a surface of the tip, and the detection DNA contains base sequences that can be complementarily bound to target DNA; and a printing substrate to which probe DNA is immobilized, wherein the probe DNA contains base sequences that can be complementarily bound to the target DNA.

The detection DNA according to the present invention contains base sequences that complementarily binds to the target DNA, and may contain an amine group at a 5′-terminal.

Also, the detection DNA may contain base sequences that are complementary to a remaining part of a target DNA after the target DNA forms a DNA/DNA dimer with probe DNA. The base sequences of the detection DNA may be affected by the secondary structure of the target DNA. Also, the base sequences and the length of the detection DNA may be affected by the melting temperature (Tₘ) of the dimer between the target DNA and the probe DNA as well as the content of guanine or cytosine in the dimer.

The printing substrate including the probe DNA printed thereon according to the present invention may be selected from the group consisting of glass, metals, plastics, silicon, silicates, ceramic, semiconductors, synthetic organic metals, synthetic semiconductors, and alloys, but the constituent material thereof is not limited to those listed above. The printing substrate including the probe DNA printed thereon is prepared by etching a pattern with an area of several tens or several hundreds of square micrometers and a depth of several nanometers on the substrate surface and then printing a probe DNA spot on the pattern. The pattern etched on the printing substrate may have an area of 10 to 900 μm² and a depth of 10 to 900 nm, but the area and depth of the pattern are not limited to those listed above and may be adjusted according to a purpose of analysis. The pattern is used for marking the location of a probe DNA spot, which would otherwise be difficult to find through an optical microscope.

A drop of an aqueous probe DNA solution is applied to the printing substrate including an etched pattern with a certain size thereon so that the probe DNA is uniformly immobilized in the form of a spot onto the substrate. The probe DNA spot may have a diameter ranging from 100 nm to 10 μm, and preferably has a diameter of fewer than 2 μm, but the size thereof is not limited to it.

The probe DNA according to the present invention may contain an amine group at a 3′-terminal thereof.

The quantitative analysis kit according to the present invention may further include an atomic force microscope.

Also, the present invention provides a method of manufacturing a printing substrate for quantitatively analyzing target DNA, the method including: a process of preparing a printing substrate, a process of etching a pattern with an area of 10 to 900 μm² and a depth of 10 to 900 nm on the printing substrate, and a process of printing a probe DNA spot by applying a drop of an aqueous probe DNA solution containing base sequences capable of complementarily binding to the target DNA on the etched pattern.

The method of manufacturing a printing substrate for quantitatively analyzing target DNA according to the present invention may further include a process of amine functionalization onto the substrate surface after etching the substrate; and a process of amine functionalization, a bifunctional linker that forms a covalent bond with the amine functional group. In one exemplary embodiment of the present invention, the linker is disuccinimidyl carbonate (DSC), but the type of the linker is not limited to it.

The printing of the probe DNA according to the present invention may be a process of dropping an aqueous probe DNA solution forming a probe DNA spot with a diameter of 100 nm to 10 μm on the printing substrate, and the probe DNA may contain an amine group at a 3′-terminal thereof for immobilization onto the printing substrate.

The printing substrate may be selected from the group consisting of glass, metals, plastics, silicon, silicates, ceramic, semiconductors, synthetic organic metals, synthetic semiconductors, and alloys, but the constituent material(s) thereof is/are not limited to those listed above.

Also, the present invention provides a method of quantitatively analyzing target DNA, the method including: (a) a hybridization process that forms DNA/DNA dimers by applying a sample containing target DNA on a printing substrate that includes probe DNA printed thereon, wherein the probe DNA contains base sequences capable of complementarily binding to the target DNA; (b) a process of bringing a cantilever for an atomic force microscope, which includes detection DNA containing base sequences capable of complementarily binding to the target DNA, into contact with the printing substrate including the DNA/DNA dimers formed thereon to conduct adhesion force mapping within an individual probe DNA spot; and (c) a process of detecting the presence of the DNA/DNA dimers, which were formed between the probe DNA and target DNA, in a spot where an adhesion force is observed on the adhesion force map, and analyzing the spatial distribution of the captured DNAs.

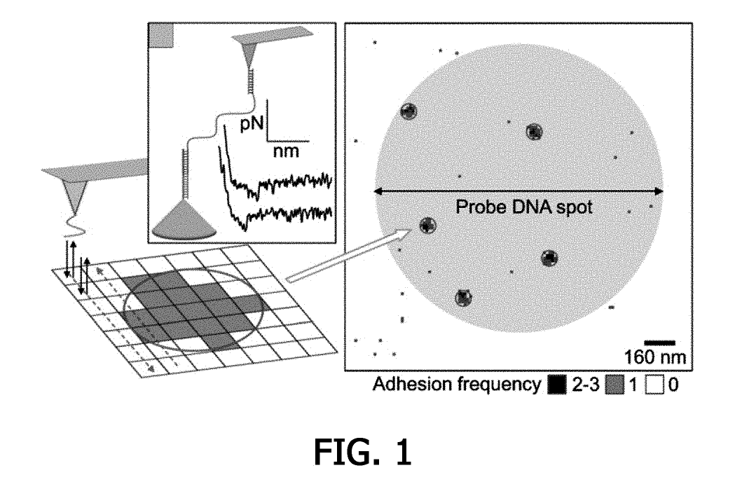

FIG. 1 is a schematic diagram for illustrating a method of quantitatively analyzing target DNA using an atomic force microscope according to one exemplary embodiment of the present invention.

As shown in FIG. 1, target DNA is applied to a probe DNA spot to form a DNA/DNA hybrid, and then adhesion force mapping is carried out by moving a cantilever, which includes detection DNA immobilized thereon, across an entire region of the printed probe DNA spot. This way, a specific force-distance curve is detected when the cantilever moves across locations of dimers of probe DNA and target DNA, and the presence and location of the target DNA may be identified based on locations of an adhesion force.

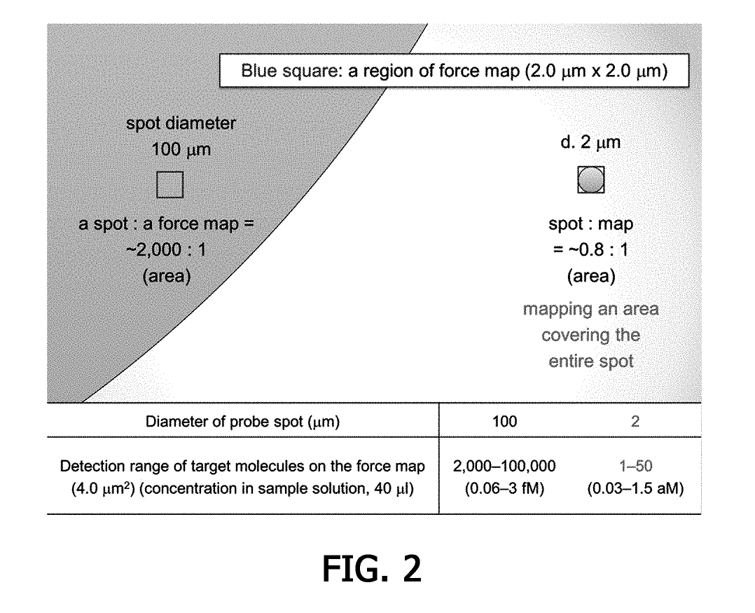

FIG. 2 is a schematic diagram for illustrating the detection range of target DNA calculated based on a probe DNA spot diameter according to one exemplary embodiment of the present invention.

As shown in FIG. 2, the image of a 2 μm×2 μm region at a resolution of 128×128 pixels gives a spatial resolution smaller than 20 nm, which is sufficient for detecting an individual target DNA molecule. Since the AFM imaging of an entire probe DNA spot with a diameter of about 100 μm under the nanometer resolution was impossible in the past, the total number of target DNA molecules was determined by scanning only a part of a probe DNA spot and estimated with respect to the entire spot area. In this case, the detection limit of DNA is as high as several femtomolar (fM) concentration. However, when the probe DNA spot diameter is within 2 μm, the entire spot area can be scanned, and thus absolute DNA quantification is possible, and detection at the attomolar (aM)-scale or less is possible without missing a single DNA molecule.

The method of quantitatively analyzing target DNA according to the present invention includes a hybridization process that forms a DNA/DNA dimer by applying a sample containing target DNA on a substrate including probe DNA printed thereon, wherein the probe DNA contains base sequences capable of complementarily binding to the target DNA.

A target DNA solution is loaded in a hybridization chamber in the form of a glass slide including a reservoir. The reservoir contains several tens of microliters. A substrate including a probe DNA spot printed thereon is covered on the hybridization chamber. When the assembly of the hybridization chamber and the substrate is vertically rotated for effective hybridization at a certain time and temperature, and thereby target DNA can be captured in the probe DNA spot region.

The sample containing target DNA according to the present invention may contain the target DNA at a concentration of 1 aM or less, and preferably contains the target DNA at a concentration of 10 zM to 1 aM.

In this case, the base sequence of the probe DNA may be selected in such a way that the melting temperature of a dimer formed between the target DNA and the probe DNA is higher than the melting temperature of a dimer formed between the target DNA and the detection DNA. In this case, the regions where probe DNA and detection DNA bind to the target DNA do not coincide and are apart by a sufficient distance there between. Therefore, when the target DNA captured on the probe DNA spot is pulled vertically by the hybridization with a detection DNA applied onto the cantilever, the rest of a single strand part of the target DNA is not involved in hybridization may be stretched elastically. In this way, a force-distance curve showing a specific stretching distance and a specific adhesion force may be obtained, and such a force-distance curve is used to distinguish specific binding from non-specific binding.

Next, the method of quantitatively analyzing target DNA according to the present invention includes a process of bringing a cantilever for an atomic force microscope, which includes detection DNA containing base sequences capable of complementarily binding to the target DNA, into contact with the printing substrate including DNA/DNA dimers formed thereon to conduct adhesion force mapping within an individual probe DNA spot

A specific adhesion force may be found from collected force-distance curves over the spot, and an adhesion force map of a single target DNA molecule is obtained by collecting force-distance curves pixel by pixel with a few nanometer resolution from where the specific adhesion force was observed. The cluster radius is calculated on the map, and the radii from the images of individual target DNA are averaged. An optimal pixel size for scanning the entire probe DNA spot region is determined based on the measured cluster radii, so that the cantilever can detect locations of target DNA molecules on the surface without missing a single molecule. Also, the cluster radius value may be used as a threshold value for sorting out false positive images.

Next, the method of quantitatively analyzing target DNA according to the present invention includes a process of detecting the presence of the DNA/DNA dimers, which were formed between the probe DNA and target DNA, in a spot where adhesion force is observed on the adhesion force map, and analyzing the spatial distribution of the captured DNAs

The analysis of spatial distribution of the captured DNAs according to the present invention may be conducted by counting the number of DNA/DNA dimers with an observed adhesion force in the sample above.

To improve a signal-to-noise ratio in the analysis of the spatial distribution of the cpatured DNAs, the locations of dimers are determined from the result after overlaying multiple force maps which were obtained from the same probe DNA spot in a consecutive manner

Specifically, the method for counting the number of the DNA/DNA dimers includes: a process of obtaining multiple adhesion force maps from the same probe DNA spot in a consecutive manner; a process of isolating clusters in which a specific adhesion force is observed in two or more adjacent pixels on the overlaid force map; a process of isolating, among the above clusters, clusters in which the specific adhesion force is repeatedly observed twice or more in the same pixels; and a process of defining clusters with a diameter greater than the diameter of a single target DNA cluster among the pixel clusters in the overlaid maps as locations of DNA/DNA dimers, and counting the number of such clusters.

A pixel cluster from which an adhesion force refers to a group of adjacent pixels. If the cluster area is similar to the average hydrodynamic radius of the target DNA, it may be determined that a DNA/DNA dimer is present at the location. This method enables the spatial distribution analysis of the target DNA possible.

The spatial distribution analysis may produce more accurate results when the adhesion force mapping of a specific region is carried out one to ten times, preferably two to six times, and more preferably three to five times and then the results are averaged.

From now on, exemplary embodiments of the present invention will be described for an understanding of the present invention. However, the following embodiments are presented only for better understanding of the present invention and not to limit the present invention to it.

### EXAMPLE 1

### Preparation of Cantilever Including Immobilized Detection DNA

An atomic force microscope from JPK Instruments AG was used. The cantilever used was DPN Probe Type B from NanoInk Inc., which has an average spring constant of about 30 to 40 pN/nm.

To immobilize detection DNA onto the cantilever, the cantilever was cleaned, subjected to silylation, and then coated with nanocone so that an amine functional groups were uniformly introduced onto a tip surface of the cantilever. Then, disuccinimidyl carbonate (DSC) was introduced onto the tip surface as a bifunctional linker that formed a covalent bond with a primary amine in a selective manner Subsequently, the cantilever was treated with an aqueous solution of detection DNA (5′—NH₂—(CH₂)₆-GTC CAG AGT GGA GGG AGA AC-3′: SEQ ID NO: 1) containing an amine group at a 5′ -terminal thereof to prepare a cantilever including the detection DNA at the tip thereof.

### EXAMPLE 2

### Preparation of Probe DNA Spot

2-1. Etching of Glass Slide

The etching of a square pattern having a depth of 200 nm and a size of 20 μm×20 μm was carried out using an inductively coupled plasma.

2-2. Printing of Probe DNA Spot

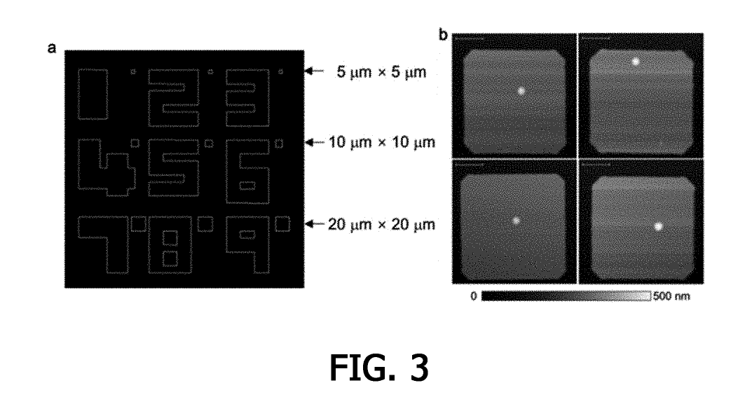

A probe DNA spot was prepared by immobilizing probe DNA onto a specific region of a solid surface. In this case, a glass slide was used as the solid surface. The glass slide was cleaned, subjected to silylation, and coated with dendron molecules so that an amine functional groups were uniformly introduced onto a tip surface thereof. Then, DSC was introduced onto the tip surface as a bifunctional linker that formed a covalent bond with a primary amine in a selective manner Subsequently, probe DNA (5′-GAC CAT CAA TAA GGA AGA AGC CCT TCA GAG GCC AGT AGC A-(CH₂)₆—NH₂-3′: SEQ ID NO: 2) containing an amine group at a 3′-terminal thereof was immobilized on the tip surface. In this case, FluidFM® from Nanosurf Inc. was used to drop an aqueous probe DNA solution on the glass slide. The cantilever includes a microchannel capable of containing an aqueous probe DNA solution (30 mM sodium citrate, 300 mM NaCl, and 12.5% glycerol). Also, there is a hole at the end of the tip with a diameter of around 300 nm to dispense the aqueous solution. The diameter of probe DNA spot was adjusted to be less than 2 μm and printed on the glass slide. An example of a probe DNA spot is illustrated in FIG. 3B. FIG. 3B is an AFM image of a 20 μm×20 μm probe DNA spot on an etched surface of a glass slide. The preparation of the probe DNA-printed glass slide was completed by immersing the glass slide in a 40° C. washing solution (30 mM sodium citrate, 300 mM NaCl, and 0.2% sodium dodecyl sulfate) for 20 minutes and rinsing off residues.

### EXAMPLE 3

### Quantitative Analysis of Target DNA

3-1. Hybridization of Target DNA

Base sequences (5′-TGC TAC TGG CCG CTG AAG GGC TTC TTC CTT ATT GAT GGT CAG CGG AAT GCT GTG GAC AGT CTG GAG TTT CAC ACA CGA GTT GGT CAG CAT CTG CAG CTC CAC GGA TGT CAG GGA GAA GCT TCT GAA ACA CTT CTT CTG CTG CTC CCG GAT GTT CTC CCT CCA CTC TGC AC-3′: SEQ ID NO: 3) from a part of an BCR-ABL gene, which is a biomarker of chronic myelogenous leukemia, were synthesized as the target DNA. 40 μl of the aqueous target DNA solution at a concentration of 40 zM (1 copy), 80 zM (2 copies), 200 zM (5 copies), 400 zM (10 copies), and 1 aM (24 copies), were heated at 95° C. for three minutes and then loaded on hybridization chambers, respectively. The glass slide according to the Example 2, which includes the probe DNA spot printed thereon, was covered on the hybridization chamber, and the assembly was vertically rotated at 52° C. for 24 hours to make the solution flow over the probe DNA spot. After the reaction, the glass slide was immersed in a washing buffer solution at 72° C. for 20 minutes and residues were rinsed off.

3-2. Analysis of Range of Hydrodynamic Behavior of Single Target DNA Molecule Hybridized with Probe DNA

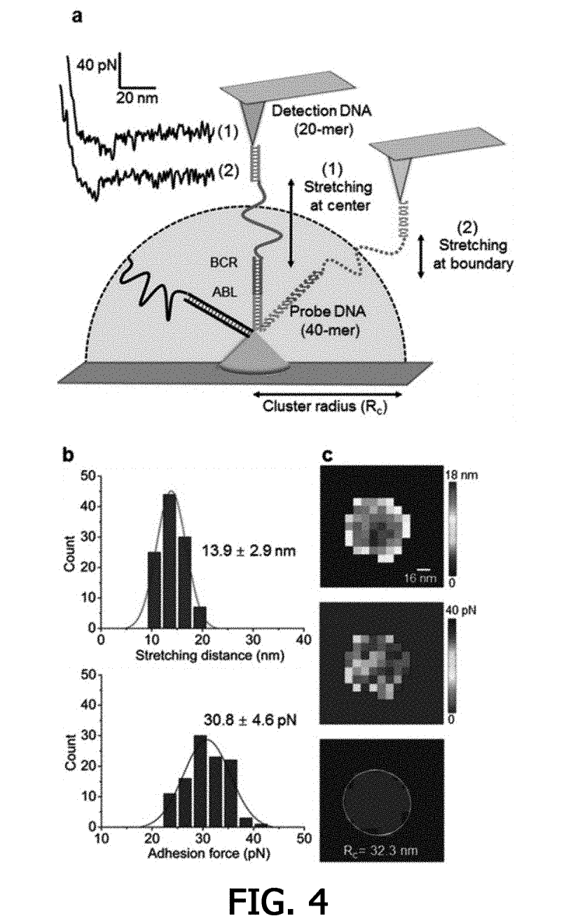

A probe DNA spot capturing target DNA was examined by an atomic force microscope in an aqueous phosphate buffered saline (PBS) solution with a cantilever according to Example 1, which includes detection DNA immobilized thereon, and a schematic diagram thereof is shown in FIG. 4A. A specific force-distance curve showing an interaction between the detection DNA and the target DNA was observed when the cantilever contacted the target DNA. At the location, an adhesion force map was obtained with a resolution of 8 nm to analyze the hydrodynamic radius of target DNA and the specific force-distance curves.

FIG. 4B is the histograms of adhesion force and the stretching distance measured from the force-distance curves showing the adhesion between the detection DNA and the dimer of target DNA and probe DNA.

In FIG. 4B, the histograms of adhesion force and the stretching distance were fitted into the Gaussian function. The values of adhesion force value and the stretching distance were 30.8 pN and 13.9 nm, respectively, and these values represented the adhesion with a part of the BCR-ABL gene. Based on the shape of a force-distance curve, a specific adhesion force can be distinguished from a nonspecific adhesion force.

As shown in FIG. 4C, the adhesion force, stretching distance, and results of ellipsoid fitting of an image of a single target DNA molecule are displayed on maps in a quantitative manner Ellipsoid fitting is a method for measuring a cluster radius of a single target DNA. Specifically, three or six images were obtained for each location. Then, three images obtained in a consecutive manner were overlaid to collect the pixels where the force was observed once or twice. Five overlaid images were obtained at the three different locations, in which case, the average cluster radius was 29.9 nm. Therefore, the location of a single target DNA was determined based on the average cluster radius, and further analysis was conducted with the method.

3-3. Analysis of Adhesion Force Map in Probe DNA Spot Treated with Various Concentrations of Target DNA

Considering the number of pixels in an adhesion force map (128×128 pixels) obtained by an atomic force microscope, the scan range covering the entire probe DNA spot was set within a range of 2.00 μm×2.00 μm to 2.35 μm×2.35 μm to attain a resolution of 15.6 nm to 18.4 nm, which is sufficiently smaller than the hydrodynamic radius of a single target DNA molecule determined in Example 3-2. For this reason, the target DNA on the map can be detected without being missed. Three adhesion force maps were obtained from the same probe DNA spot in a consecutive manner with the resolution.

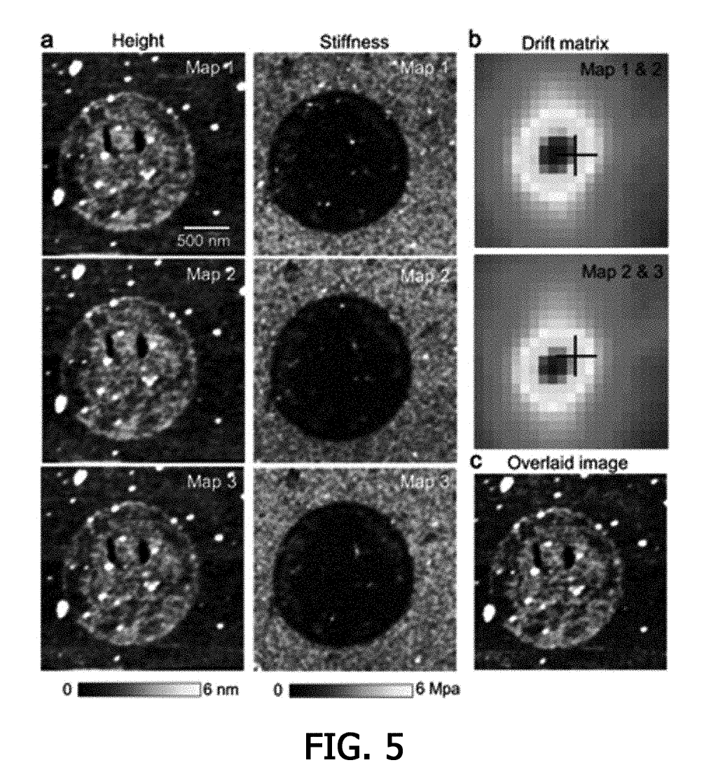

The three adhesion force maps were overlaid to improve the signal to noise ratio. First, “height” images and “Young's modulus” images of the probe DNA spot, which can be simultaneously obtained with adhesion force maps, were analyzed, the degree of image drift along the time was determined by comparing the first and second images and the second and third images, and was adjusted to the images. As a result, a probe DNA spot image was obtained after drift adjustment and overlaying process as shown in FIG. 5.

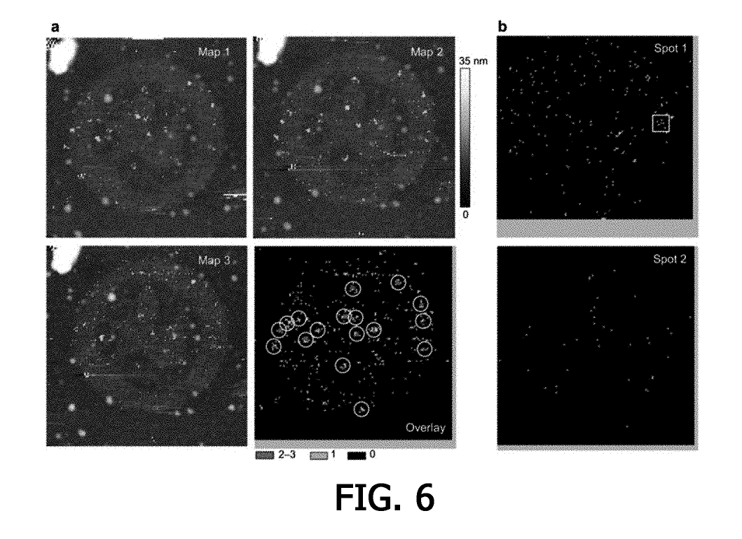

The adhesion force maps of 1 aM target DNA as shown in FIG. 6A are three adhesion force maps obtained in a consecutive manner and a cluster obtained by overlaying the adhesion force maps. Pixels from which an adhesion force was observed once appear green, and pixels from which the adhesion force was observed twice or more appear red on the map. The number of clusters from which the adhesion force was observed twice or more and having a radius of 26.0 nm or more was 17. In contrast, in an adhesion force map obtained from a probe DNA spot as a negative control shown in FIG. 6B, only one cluster was observed when nine spots were examined

Next, adhesion force mapping of a spot containing hybridized target DNA at a concentration other than 1 aM was conducted, and results thereof were analyzed. Target DNA contained at a concentration of 0 M, 40 zM, 80 zM, 200 zM, and 400 zM, respectively, was subjected to hybridization within a probe DNA spot and was repeatedly analyzed through adhesion force mapping. In the case of target DNA contained at a very low concentration of 40 zM, nine different analyses were conducted in consideration of various conditions that may affect quantification. In the case of higher target DNA concentrations, five or three different analyses were conducted. Only the clusters with a radius greater than 26.0 nm were counted when determining the number of clusters, and results thereof are provided in the following Table 1.

TABLE 1
Number of clusters observed at various target DNA concentrations

| Target | Theoretical | | | | | | | | | | | |
|---|---|---|---|---|---|---|---|---|---|---|---|---|
| DNA | number of | Number of clusters observed at each spot | | | | | | | | | | |
| concentration | DNA | (when cluster radius is 26.0 nm or more) | | | | | | | | | | Standard |
| (zM) | molecules | #1 | #2 | #3 | #4 | #5 | #6 | #7 | #8 | #9 | Mean | deviation |
| 0 | 0 | 0 | 0 | 0 | 0 | 0 | 0 | 0 | 0 | 0 | | |
| 40 | 1 | 0 | 0 | 0 | 1 | 0 | 0 | 0 | 0 | 1 | 0.2 | 0.44 |
| 80 | 2 | 1 | 0 | 0 | 2 | 0 | — | — | — | — | 0.6 | 0.89 |
| 200 | 5 | 3 | 2 | 2 | 3 | 2 | — | — | — | — | 2.4 | 0.55 |
| 400 | 10 | 6 | 3 | 5 | — | — | — | — | — | — | 4.7 | 1.53 |

At a concentration of 40 zM, which corresponds to the presence of only one target DNA molecule, one cluster was observed in each of two spots out of nine spots. At a concentration of 80 zM corresponding to the presence of two target DNA molecules, each of two spots out of five spots were found to contain one cluster and two clusters, respectively. When five (i.e. 200 zM) or ten (i.e. 400 zM) target DNA molecules were present, all spots were observed to contain one or more clusters. In this case, the average number of clusters observed in each spot was 2.4 for 200 zM and 4.7 for 400 zM.

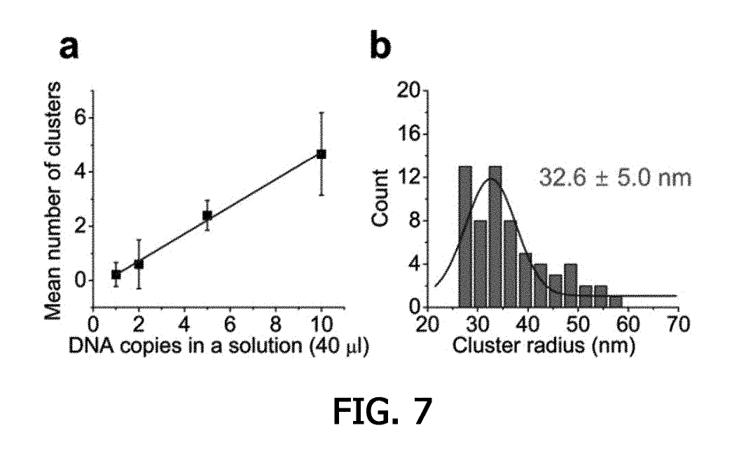

FIG. 7 shows the correlation between the number of target DNA molecules in a sample and the number of clusters according to analysis results. The above two values show a strong linear correlation (linear regression model, R²=0.994), and the average cluster is 32.6 nm. The results confirm that DNA at a very low concentration can be detected without DNA amplification and transformation or fluorescent marker.

What is claimed is:

1. A kit for quantitatively analyzing target DNA, the kit comprising:
a cantilever for an atomic force microscope, the cantilever including a tip at the end of a body thereof and detection DNA immobilized to a surface of the tip, wherein the detection DNA contains base sequences that can be complementarily bound to target DNA; and
a printing substrate to which probe DNA is immobilized, wherein the probe DNA contains base sequences that can be complementarily bound to the target DNA.

2. The kit according to claim 1, wherein the probe DNA is printed as a spot with a diameter of 100 nm to 10 μm on the printing substrate.

3. The kit according to claim 1, wherein the probe DNA contains an amine group at a 3′-terminal thereof.

4. The kit according to claim 1, wherein the printing substrate is selected from the group consisting of glass, metals, plastics, silicon, silicates, ceramic, semiconductors, synthetic organic metals, synthetic semiconductors, and alloys.

5. The kit according to claim 1, wherein the detection DNA contains an amine group at a 5′-terminal thereof.

6. The kit according to claim 1, further comprising an atomic force microscope.

7. A method of manufacturing a printing substrate for quantitatively analyzing target DNA, the method including:
preparing a printing substrate;
etching a pattern with an area of 10 to 900 μm² and a depth of 10 to 900 nm on the printing substrate; and
printing a probe DNA spot by applying, on the etched pattern, a drop of an aqueous probe DNA solution containing base sequences capable of complementarily binding to the target DNA.

8. The method according to claim 7, wherein the printing of probe DNA is a process of dropping an aqueous probe DNA solution forming a probe DNA spot with a diameter of 100 nm to 10 μm on the printing substrate.

9. The method according to claim 7, wherein the probe DNA contains an amine group at a 3′-terminal thereof.

10. The method according to claim 7, wherein the printing substrate is selected from the group consisting of glass, metals, plastics, silicon, silicates, ceramic, semiconductors, synthetic organic metals, synthetic semiconductors, and alloys.

11. The method according to claim 7, further comprising:
amine functionalization onto the substrate surface after etching the substrate; and
introducing, to the amine functional group, a bifunctional linker that forms a covalent bond with the amine functional group in a selective manner

12. A method of quantitatively analyzing target DNA, the method including:
(a) hybridizing that forms DNA/DNA dimers by applying a sample containing target DNA into contact with a printing substrate that includes probe DNA printed thereon, wherein the probe DNA contains base sequences capable of complementarily binding to the target DNA;
(b) bringing a cantilever for an atomic force microscope, which includes detection DNA containing base sequences capable of complementarily binding to the target DNA, into contact with the printing substrate including the DNA/DNA dimers formed thereon to conduct adhesion force mapping within a probe DNA spot; and
(c) detecting the presence of the DNA/DNA dimers, which were formed between the probe DNA and the target DNA, in a spot where an adhesion force is observed on the adhesion force map, and analyzing the spatial distribution of the captured DNAs.

13. The method according to claim 12, wherein sample containing target DNA contains the target DNA at a concentration of 10 zM to 1 aM.

14. The method according to claim 12, wherein the printing substrate that includes probe DNA printed thereon includes the target DNA immobilized in the form of a spot with a diameter of 100 nm to 10 μm onto a surface thereof.

15. The method according to claim 12, wherein the adhesion force mapping according to (b) is conducted by measuring a specific force-distance curve obtained as a result of an interaction between the detection DNA and the target DNA, which takes place where the cantilever for an atomic force microscope contacts the target DNA.

16. The method according to claim 12, wherein the analysis of spatial distribution of the captured DNAs according to (c) is conducted by counting DNA/DNA dimers with an observed adhesion force in the sample containing the target DNA.

17. The method according to claim 16, wherein the counting the number of the captured DNAs with an observed adhesion force includes:
obtaining multiple adhesion force maps from the same probe DNA spot in a consecutive manner;
isolating clusters in which a specific adhesion force is observed in two or more adjacent pixels after overlaying multiple adhesion force maps;
isolating, among the clusters, clusters in which the specific adhesion force is repeatedly observed twice or more in the same pixels; and
counting, among pixel clusters resulting from overlaying multiple pixels, clusters with a diameter greater than a diameter of a single target DNA cluster.

18. The method according to claim 12, further comprising determining a concentration of the target DNA after (c).


SEQUENCE LISTING (3 sequences)

SEQ ID NO: 1 - DNA, 20 bp, Artificial
  detection DNA
gtccagagtggagggagaac

SEQ ID NO: 2 - DNA, 40 bp, Artificial
  probe DNA
gaccatcaataaggaagaagcccttcagaggccagtagca

SEQ ID NO: 3 - DNA, 170 bp, Artificial
  BCL-ABL gene
tgctactggccgctgaagggcttcttccttattgatggtcagcggaatgctgtggacagtctggagtttcacacacgagttggtcagcatctgcagctccacggatgtcagggagaagcttctgaaacacttcttctgctgctcccggatgttctccctccactctgcac